(19) United States
(12) Patent Application Publication
(10) Pub. No.: US 2023/0200697 A1
(43) Pub. Date: Jun. 29, 2023

(54) AUTOMATIC EVOLUTION METHOD FOR BRAINWAVE DATABASE AND AUTOMATIC EVOLVING SYSTEM FOR DETECTING BRAINWAVE

(71) Applicant: ExeBrain Co., Ltd., Taipei City (TW)

(72) Inventors: Shu-Chun Kuan, Taipei City (TW); Jason Chun-Cheng Lin, Taipei City (TW); Chin-Yeh Lu, Taipei City (TW)

(21) Appl. No.: 17/886,600

(22) Filed: Aug. 12, 2022

(30) Foreign Application Priority Data
Dec. 24, 2021 (TW) 110148782

(51) Int. Cl. A61B 5/16; A61B 5/00

(52) U.S. Cl. CPC A61B 5/165; A61B 5/7267; A61B 5/24

(57) ABSTRACT

An automatic evolution method used for a brainwave database which collects physiological information of brainwaves about healthy and clinical groups, the automatic evolution method includes: classifying the physiological information of brainwaves collected by the brainwave database according to data characteristics; establishing a feedback algorithm model based on a neural network architecture according to the physiological information of brainwaves classified by the parameters; using the feedback algorithm model to input a subject's physiological information of brainwaves; measuring an accuracy of the subsequent performance data calculated by the feedback algorithm model; and incorporating the physiological information of brainwaves of the subject into the brainwave database, establishing an updated feedback algorithm model based on an updated neural network architecture, and feeding a comparison result generated by the updated feedback algorithm model back to the subject.

### CROSS-REFERENCE TO RELATED APPLICATION

This application claims the priority of Taiwanese patent application No. 110148782, filed on Dec. 24, 2021, which is incorporated herewith by reference.

### BACKGROUND OF THE INVENTION

### 1. Field of the Invention

The present invention relates to a system and method for detecting brainwaves, and particularly to an automatic evolution brainwave detection system and an automatic evolution method used for brainwave database.

### 2. The Prior Arts

Existing biofeedback training mainly uses a wireless device at the input terminal, such as a pair of electrode pads to compare the variation of brainwave before and after training in three regions of the parietal lobe, and a pair of electrode pads to detect the influence of neurofeedback for sensorimotor rhythm (SMR), or collect physiological signals and upload the physiological data to the cloud platform for analysis through wired or wireless transmission modules, the individual needs to open the APP or related applications to read data of the physiological device during the sleep period in a retrospective manner. However, in the prior art, subjects usually cannot obtain physiological information such as brainwaves or heart rate variability immediately, and need to wait several hours to several days for interpretation.

Meanwhile, although the existing smart bed group health management system also collects physiological signals, the physiological signals is collected when the individual is sleeping in bed. The physiological signals are uploaded to the cloud platform for analysis through wired or wireless transmission modules, the subject needs to open relevant applications to read the physiological devices during sleep in a retrospective manner. The disadvantage is that the physiological signals of the subject cannot be calculated immediately after transmission, the feedback cannot be transmitted to the subject.

In addition, although there is a feedback mechanism of functional magnetic resonance imaging (real time fMRI neurofeedback), the magnetic resonance imaging equipment is quite expensive thus mostly installed in medical institutions, and it takes more than 30 minutes to collect the signal and perform the imaging process. The calculation of the feedback mechanism also takes more than 10 minutes, and the remote home configuration and immediate (within 1 minute) analysis feedback cannot be achieved.

Therefore, it is necessary to provide an improved method and system that can provide real-time feedback at the remote terminal, so that the subject can immediately understand the conditions, and the subject can adjust their physiological signals for recovery through visual or auditory feedback.

### SUMMARY OF THE INVENTION

In order to effectively solve the above problems, the present invention provides an automatic evolution method used for brainwave database which collects physiological information of brainwaves about healthy and clinical groups, the automatic evolution method includes: classifying the physiological information of brainwaves collected by the brainwave database according to data characteristics with a training device; establishing a feedback algorithm model based on a neural network architecture according to the physiological information of brainwaves classified by the parameters with the training device; inputting the physiological information of brainwaves of a subject through the feedback algorithm model to calculate a subsequent performance data related to the physiological information of brainwaves with an evaluation and prediction device; measuring an accuracy of the subsequent performance data calculated by the feedback algorithm model with the evaluation and prediction device to verify an evaluation index of the feedback algorithm model according to the known physiological information of brainwaves of the subject; and incorporating the physiological information of brainwaves of the subject into the brainwave database, establishing an updated feedback algorithm model based on an updated neural network architecture, and feeding a comparison result generated by the updated feedback algorithm model back to the subject with the evaluation and prediction device.

According to an embodiment of the present invention, the physiological information of the brainwaves includes gender, age, education level, mental state and behavioral feature.

According to an embodiment of the present invention, wherein the mental state and behavioral feature included in the classified brainwave physiological information includes emotional states, cognitive functions, and personality characteristics.

According to an embodiment of the present invention, wherein the mental state and behavioral feature included in the classified brainwave physiological information includes memory, sleep disorder, anxiety, depression and personality characteristics, among which anxiety and depression belong to the mental state.

According to an embodiment of the present invention, the physiological information of brainwaves corresponds to a behavioral performance and a mental process of the subject.

The present invention further provides an automatic evolution brainwave detection system including: a brainwave database collecting physiological information of brainwaves about healthy populations and clinical populations;

a training device for executing a training step, the training step including classifying the physiological information of brainwaves collected by the brainwave database according to data characteristics, so as to establish a feedback algorithm model; and

an evaluation and prediction device coupled to the brainwave database for performing a plurality of steps including: using the feedback algorithm model to input the physiological information of brainwaves of a subject to calculate a subsequent performance data related to the physiological information of brainwaves; measuring an accuracy of the subsequent performance data calculated by the feedback algorithm model according to the known physiological information of brainwaves of the subject to verify an evaluation index of the feedback algorithm model; and incorporating the physiological information of brainwaves of the subject into the brainwave database, establishing an updated feedback algorithm model; and a feedback device generating a feedback signal to the subject by using the updated feedback algorithm model.

The detection and comparison of the brainwave or physiological signals database of the present invention uses artificial intelligence-related machine learning, so the feedback algorithm model can evolve with the increase of the detection of the subject, and the real-time feedback at the remote terminal can be used for the subject. The subjects can immediately (for example, within 1 minute) understand the conditions, and the subject can adjust their physiological signals for recovery through visual or auditory feedback.

### BRIEF DESCRIPTION OF THE DRAWINGS

FIG. 1 is a block diagram of a brainwave detection system according to an embodiment of the present invention;

FIG. 2 is a schematic diagram of an automatic evolution method according to an embodiment of the present invention;

FIG. 3 is a schematic diagram of a feedback algorithm model according to an embodiment of the present invention;

FIG. 4 is an evolution curve diagram of a feedback algorithm model according to an embodiment of the present invention; and

FIG. 5 and FIG. 6 are schematic diagrams of types of corresponding behavioral performance and mental process according to an embodiment of the present invention.

### DETAILED DESCRIPTION OF THE PREFERRED EMBODIMENT

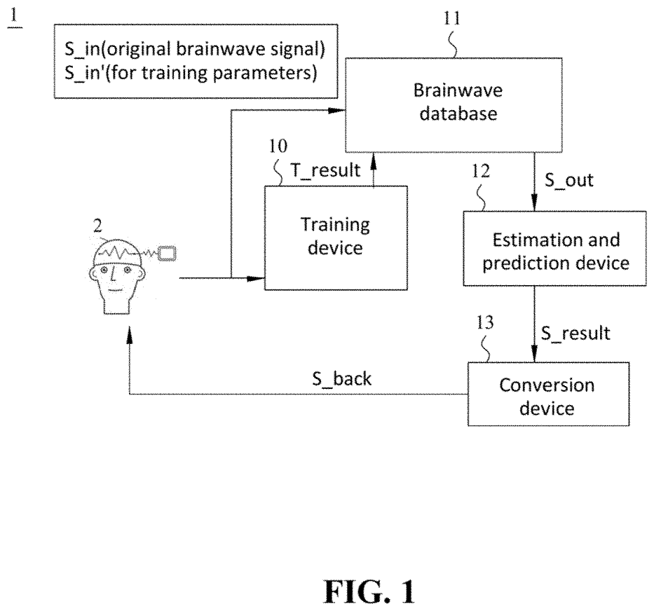

Please refer to FIG. 1, FIG. 1 is a block diagram of a brainwave detection system according to an embodiment of the present invention, as shown in FIG. 1, the present invention provides an automatic evolution brainwave detection system 1, which includes a training device 10, a brainwave database 11, an evaluation and prediction device 12, and a conversion device 13. The training device 10 executes a training step. The training step includes classifying the input data S_in according to the characteristics of the input data S_in of the subject 2, so as to obtain a feedback algorithm model T_result. The brainwave database 11 is coupled to the training device 10 for storing the input data S_in and the feedback algorithm model T_result, and outputting an output signal S_out. The estimation and prediction device 12 is coupled to the brainwave database 11, and is used for performing a plurality of steps including: receiving the output signal S_out to estimate a subsequent data S_result according to the feedback algorithm model T_result; evaluating the feedback algorithm model T_result and the subsequent performance data S_result and using the feedback algorithm model T_result to generate a feedback signal S_back through the conversion device 13.

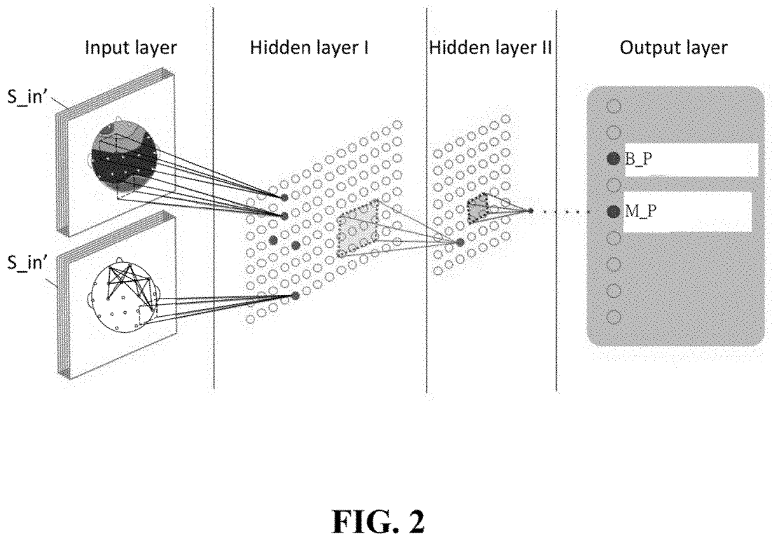

Please refer to FIG. 2, FIG. 2 is a schematic diagram of an automatic evolution method according to an embodiment of the present invention, as shown in FIG. 2, the training device 10 used in the training step includes a brainwave cap and software and hardware equipment. The training device has the following functions: (1) transmitting the original brainwave signals to the brainwave database, (2) in the training device, calculating the brainwave signals collected by the brainwave cap through a digital signal processor (DSP) or central processing unit (CPU), then identifying the brainwave pattern through state analysis, and performing the setting process of the parameters of the neurofeedback in the most efficient way. The training step includes classifying the input data S_in, S_in′ according to the characteristics of the input data S_in, S_in′, and the classified parameter includes gender, age, education level, mental state and behavioral feature. In a preferred embodiment of the present invention, the mental state and behavioral feature include emotional state, cognitive function, and personality traits. In yet another embodiment of the present invention, the mental state and behavioral features include memory, sleep disorder, anxiety, depression and personality characteristics wherein anxiety and depression belong to emotional states. The basic data of gender, age and education level are parameters to be compared in the brainwave database. The following method can improve the accuracy: (1) The original 500×500 matrix data or brainwave graphics needed to be transmitted can be replaced by 400×400 matrix data or brainwave graphics through the automatic evolution of the database to describe current status of brain function; and (2) the original 500×500 matrix data or brainwave graphics can also be replaced with another 500×500 matrix data or brainwave graphics to reflect the current state of brain function more accurately, and to obtain the algorithm model of an input layer, a hidden layer, and an output layer wherein the input layer is part of the input data S_in or S_in′. The hidden layer shown in the embodiment of FIG. 2 has two or more layers, but through the automatic evolution algorithm of the database in this case, the state of brainwave function can also be depicted through only one hidden layer, that is, there may be more than one hidden layer between the input layer and the output layer. The purpose is to find the optimal prediction mode of the brainwave corresponding to behavioral performance B_P or mental process M_P. The input data S_in mainly is provided to the brainwave database, and the input data S_in′ is mainly provided to the comparison of neurofeedback training. The evaluation and prediction device 12 in FIG. 1 can complete the calculation in the cloud, and the conversion device 13 is implemented by software and hardware at the user terminal. The hardware device on the user side contains a CPU, DSP, GPU, or TPU to process brainwave thresholds or graphics. The core technology of the present invention is automatic evolution, that is, the processing and calculation of brainwave signals can be updated and evolved with the increase of data. In the output layer, the behavioral performance B_P and the mental process M_P described by the algorithm model will be output, the behavioral performance B_P and the mental process M_P corresponding to the brainwave signals contained in the input data S_in and S_in′ refer to the explicit behavior or internal performance, explicit behaviors such as observable behaviors like attention, memory, problem solving, knowledge, body movement performance, etc. The internal performance includes internal operation such as state of consciousness, positive and negative emotions, sleep, hallucinations or delusions . . . etc. The behavioral performance B_P and the mental process M_P can be presented in a digital manner through brainwaves or other biological signals.

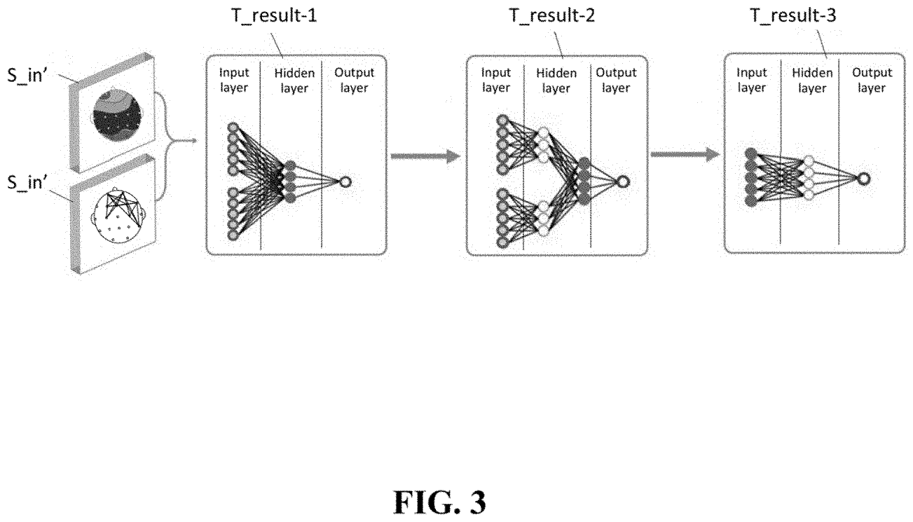

Please refer to FIG. 3, FIG. 3 is a schematic diagram of a feedback algorithm model according to an embodiment of the present invention, as shown in FIG. 3, in this embodiment, the brainwave database 11 includes at least one feedback algorithm model, and each algorithm model evolves as the usage of the subject increases or the frequency of use increases. That is, the training step generates an updated feedback algorithm model according to the input data and the at least one feedback algorithm model. The embodiment of FIG. 3 shows that there is one hidden layer after calculation, but after algorithm analysis and feature extraction in this case, there may also be two or more hidden layers to describe the functional state of brainwaves, that is, there may be more than one hidden layer between the input layer and the output layer. For example, the algorithm model T_result−1 will adjust the according to the same behavioral performance B_P or the mental process M_P to generate the algorithm model T_result−2. The adjustment is based on machine learning and deep learning and using massive data distributed storage, graphics processing unit (GPU) or tensor processing unit (TPU), etc., through linear regression, random forest, multilayer perceptron, deep neural network, convolutional neural network or recursive neural network and other model architectures to perform feature extraction, classification, and grouping of brainwave data. Then, as the data of the subject data gradually increases, the feedback algorithm model T_result−2 will adjust according to the same behavioral performance B_P or mental process M_P to generate the feedback algorithm model T_result−3.

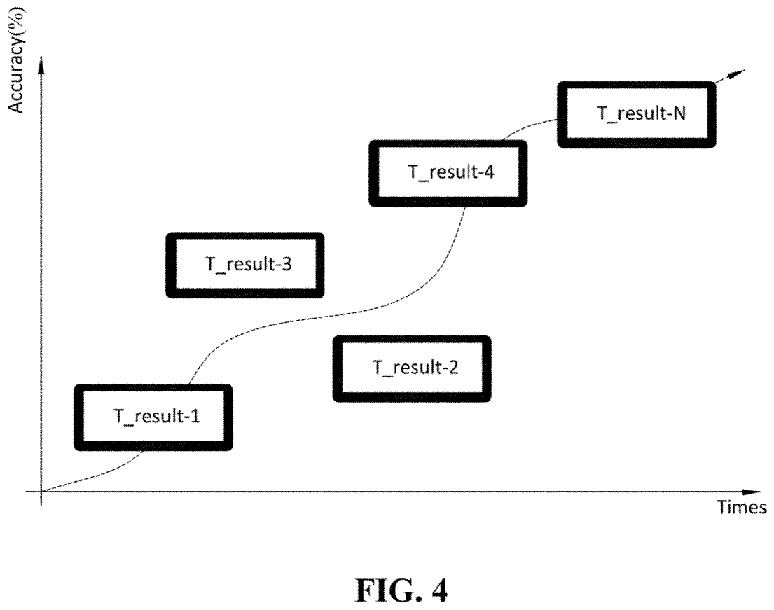

Please refer to FIG. 4, FIG. 4 is an evolution curve diagram of a feedback algorithm model according to an embodiment of the present invention, the evaluation and prediction device 12 receives the output signal to calculate a follow-up data according to the feedback algorithm model, and evaluate the accuracy of the feedback algorithm model and the subsequent data. As shown in FIG. 4, as the number of times of use by the subject increases, the feedback algorithm model T_result−1 evolves to the feedback algorithm model T_result−N, and the corresponding accuracy also continues to improve.

For example, a subject with an insomnia problem can improve the insomnia problem through neurofeedback. The subject is initially compared with an initial brainwave database, the conversion device 13 is included in the training device, and the training device divides the brainwave signal into the input data S_in and the input data S_in′ and sends the input data S_in and the input data S_in′ to the brainwave database, the input data S_in is the original brainwave signal, and the input data S_in′ is the converted brainwave pattern and characteristics. The conversion device 13 generates a feedback signal S_back according to the feedback algorithm model T_result−1 because the feedback algorithm model T_result−1 is the most suitable model in the initial stage. The present invention mainly emphasizes automatic evolution model calculation, so the model at each stage is calculated by machine learning and deep learning (or artificial intelligence). As the number of brainwaves on the subject gradually increases, it will gradually evolve from the feedback algorithm model T_result−1 to the feedback algorithm model T_result−2 or the feedback algorithm model T_result−N. The evolution from the feedback algorithm model T_result−1 to the feedback algorithm model T_result−2, or from the feedback algorithm model T_result−N to the feedback algorithm model T_result−(N+1) needs several brainwave signal input before automatic evolution, the feedback algorithm model needs to operate to perform the estimation. FIG. 3 and FIG. 4 shows the same concept. FIG. 3 indicates the mode that needs to be used between the input and output layers. FIG. 4 is a schematic diagram of the automatic evolution of brainwaves, the number of times refers to the increase in the number of subjects or the increase in brainwave data, which is used to calculate the most suitable or optimized model. For example, as the number of subjects with insomnia problems increases, the input data of the subjects also increases, the brainwave database and the feedback algorithm model automatically evolves, and when the new subject improves insomnia through neurofeedback, the conversion device 13 generates a feedback signal according to the feedback algorithm model T_result−2 because the feedback algorithm model T_result−2 at this time is most suitable for the new subject. Similarly, when the last subject improves insomnia through neurofeedback, the conversion device 13 will generate a feedback signal according to the feedback algorithm model T_result−3 because the feedback algorithm model T_result−3 at this time is most suitable for the last subject.

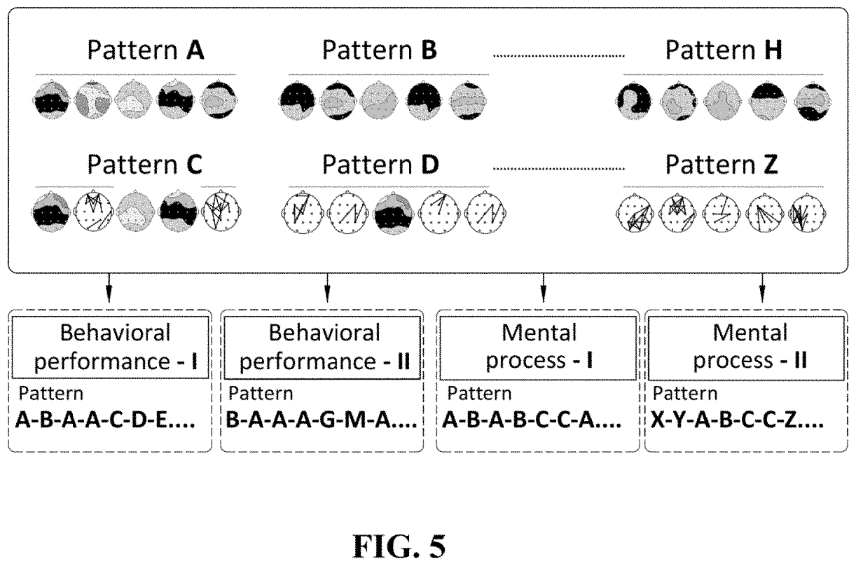

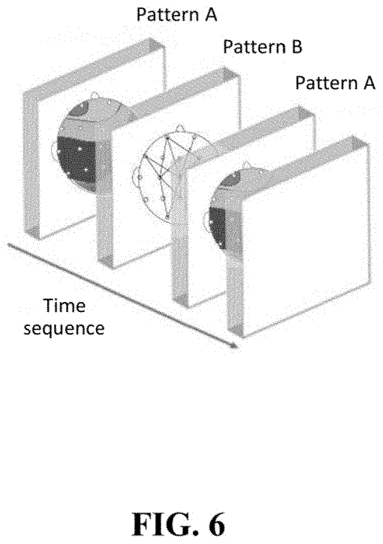

Therefore, as shown in FIG. 5 and FIG. 6, automatic evolution is to find different patterns through the brainwave data of the input data S_in, and the combination of these patterns corresponds to different behavioral performance B_P or mental process M_P, and these different behavioral performance B_P or mental process M_P needs to be described or predicted through the patterns, which needs to be calculated through machine learning or deep learning, similar to brainwave gene sequence expressed through four nucleic acids of ATCG containing nitrogenous bases, and infinite combinations of linear permutations. The automatic evolution brainwave database uses different brainwave analysis to find specific patterns. The linear or nonlinear arrangement and combination of these patterns can predict behavioral performance B_P or mental process M_P. The automatic evolution mentioned in the present invention is to classify what basic patterns are formed, and the linear and nonlinear combination of these patterns can improve the accuracy of behavioral performance B_P or mental process M_P. The brainwave training data of the input data S_in′ is to optimize training parameters of the subject and to find out the characteristic values, so as to shorten the comparison time and reduce the amount of data required and thus achieve the purpose of real-time analysis and comparison and real-time feedback.

By using the automatic evolution method of the brainwave database and the automatic evolution brainwave detection system of the present invention, with the increase of the number of subjects used and the input signal imported into the brainwave database, the feedback algorithm model can automatically evolve and make the prediction of the neurofeedback algorithm more accurate, and can also achieve the fastest transmission and the most accurate feedback with the smallest amount of data.

The present invention is not limited to the above-described embodiments, and it is obvious to those skilled in the art that various modifications and variations can be made to the present invention without departing from the spirit or scope of the present invention. Accordingly, the present invention is intended to cover modifications and variations of this invention or those falling within the scope of the appended claims and the equivalents.

What is claimed is:

1. An automatic evolution method used for a brainwave database which collects physiological information of brainwaves about healthy and clinical groups, the automatic evolution method comprising:
classifying the physiological information of brainwaves collected by the brainwave database according to data characteristics with a training device;
establishing a feedback algorithm model based on a neural network architecture according to the classified physiological information of brainwaves with the training device;
inputting the physiological information of brainwaves of a subject through the feedback algorithm model to calculate a subsequent performance data related to the physiological information of brainwaves with an evaluation and prediction device;
measuring an accuracy of the subsequent performance data calculated by the feedback algorithm model with the evaluation and prediction device to verify an evaluation index of the feedback algorithm model according to the known physiological information of brainwaves of the subject; and
incorporating the physiological information of brainwaves of the subject into the brainwave database, establishing an updated feedback algorithm model based on an updated neural network architecture, and feeding a comparison result generated by the updated feedback algorithm model back to the subject with the evaluation and prediction device.

2. The automatic evolution method of claim 1, wherein the physiological information of brainwaves includes gender, age, education level, mental state and behavioral feature.

3. The automatic evolution method of claim 1, wherein the physiological information of brainwaves corresponds to a behavioral performance and a mental process of the subject.

4. An automatic evolution brainwave detection system comprising:
a brainwave database collecting physiological information of brainwaves about healthy populations and clinical populations;
a training device for executing a training step, the training step including classifying the physiological information of brainwaves collected by the brainwave database according to data characteristics, so as to establish a feedback algorithm model; and
an evaluation and prediction device coupled to the brainwave database for performing a plurality of steps including:
using the feedback algorithm model to input the physiological information of brainwaves of a subject to calculate a subsequent performance data related to the physiological information of brainwaves;
measuring an accuracy of the subsequent performance data calculated by the feedback algorithm model to verify an evaluation index of the feedback algorithm model according to the known physiological information of brainwaves of the subject; and
incorporating the physiological information of brainwaves of the subject into the brainwave database, establishing an updated feedback algorithm model; and
a feedback device generating a feedback signal to the subject by using the updated feedback algorithm model.

5. The automatic evolution brainwave detection system of claim 4, wherein the physiological information of brainwaves includes gender, age, education level, mental state and behavioral feature.

6. The automatic evolution brainwave detection system of claim 4, wherein the physiological information of brainwaves corresponds to a behavioral performance and a mental process of the subject.

7. The automatic evolution brainwave detection system of claim 4, wherein the training step is to generate the updated feedback algorithm model according to the input data and the at least one feedback algorithm model.
